(19) United States
(12) Patent Application Publication
(10) Pub. No.: US 2009/0220517 A1
(43) Pub. Date: Sep. 3, 2009

(54) METHOD FOR PRODUCING EPITOMERS AND THEIR USES ON CARRIER MICROORGANISMS

(75) Inventors: Veit Braun, Mainz (DE); Christoph Von Eichel-Streiber, Schweppenhausen (DE); Ralf Jochem, Bad Homburg (DE)

Correspondence Address: DAVID S. RESNICK, NIXON PEABODY LLP, 100 SUMMER STREET, BOSTON, MA, 02110-2131, US

(73) Assignee: tgcBIOMICS GmbH, Mainz (DE)

(21) Appl. No.: 12/162,804

(22) PCT Filed: Nov. 29, 2006
(86) PCT No.: PCT/EP06/11461

§ 371 Date: Dec. 2, 2008

Related U.S. Application Data
(60) Provisional application No. 60/808,719, filed on May 27, 2006.

(30) Foreign Application Priority Data
Feb. 1, 2006 (DE) 10 2006 004 612.9

(51) Int. Cl. A61K 39/395; A61K 39/00; A61K 39/02; A61K 35/74; A61K 35/66; A61K 35/12; C12N 15/09

(52) U.S. Cl. 424/141.1; 424/185.1; 424/190.1; 424/235.1; 424/274.1; 424/93.4; 424/93.51; 424/93.7; 435/455; 435/471

(57) ABSTRACT

The present invention relates to a method for producing carrier microorganisms, in particular bacteria, which, through targeted genetic manipulation, carry epitopes or epitomers, respectively, on their surfaces. Epitomers are antigenically effective epitopes that can be found in the polypeptide chain in multiple identical copies, and which, when expressed on the surface of the bacteria, can be used for immunization with particular success. A further aspect relates to correspondingly produced bacteria and their uses as vaccines, in particular in cancer therapy.

The present invention relates to a method for producing carrier microorganisms, in particular bacteria, which, through targeted genetic manipulation, display epitopes or epitomers, respectively, on their surfaces. Epitomers are antigenically effective epitopes that are present in the polypeptide chain in multiple identical copies, and which, when expressed on the surface of the bacteria, can be used for the immunization with particular success. A further aspect relates to accordingly produced bacteria and their uses as vaccines, in particular in cancer therapy.

### BACKGROUND OF THE INVENTION

Vaccines are formulations that are not harmful for the vaccinated individual, but simulate the infection with an infectious agent in the body. Vaccines contain non-virulent forms of a pathogen or parts of such pathogens. Their goal is, to induce an immunological memory in the vaccination with a vaccine, which protects the vaccinated individual in case of an infection with the natural infectious agent.

For the purposes of the present invention, all cited publications are incorporated by reference in their entireties.

Most of the currently available vaccines are based on the natural form of the pathogen which was converted into an apathogenic or a weakly pathogenic form through killing, inactivation or attenuation. Alternatively, vaccines contain a selection of native antigenic components of the pathogen. The basic concept of such classical vaccines is based on a design of the vaccines in such a way that they are as similar as possible to the natural pathogens. Due to this, the immune response during a vaccination becomes very similar to the response of the immune system during a natural infection, without the existing danger of a disease. Until today, the biggest successes for the health of both humans and animals were achieved with classical vaccines. Despite this, classical vaccines are not yet suitable for fighting a series of important human diseases, such as, for example, cancer, malaria, HIV, and HCV.

Specifically for the therapy of these diseases, recently peptide-based or epitope-based vaccines were regarded as an alternative to classical vaccines. These innovative vaccines are based on the concept to confront the immune system with only one or few antigenic structures (=epitopes), and thus to trigger a very specific immune response, and to minimize side-effects that are typically found when using classical vaccines. The low immunogenicity of synthetic peptides was identified as a drawback of epitope-based vaccines, if they are administered without suitable adjuvants and carriers, because (1) the peptides are degraded in the body much too rapidly, in order to be recognized by the immune system, or (2) the peptides have no suitable T-cell epitopes being required for the induction of antibodies. Therefore, a strong interest exists, to develop safe and effective methods for the use of epitope-based vaccines that guarantee for a high immunogenicity of the epitope (McGeary et al., J. Peptide Sci, 2003, 9: 405).

Strategies for the development of peptide-based vaccines, on the one hand, attempt to improve the stability of peptides against proteolytic degradation, and thus to increase the bioavailability of the peptide antigens in the body or, on the other hand, seek to develop suitable adjuvants or carriers for the peptides (Lazoura and Apostolopoulos, Curr Med Chem, 2005, 12: 1481).

In order to improve the stability of peptides in the body, technologies for producing cyclic peptides, retro-inverso peptides, and peptides that contain non-natural components were developed.

The cyclization of peptides can be achieved through the formation of disulfide bridges via the side chains of amino acids or through cyclization of the main chain of the peptide through the formation of an amide bond (Davies, J Pept Sci, 2003, 9: 471). Compared to linear peptides and peptides that are cyclized through disulfide bridges, peptides that are cyclic via the main chain exhibit an increased stability against proteolytic degradation. Cyclic peptides were used for vaccination experiments in diseases such as multiple sclerosis and diabetes (Tselios et al, J Med Chem, 1999, 42: 1170, Dunsavage et al., J Autoimmun, 1999, 12:233, Lazoura and Apostolopoulos, Curr Med Chem, 2005, 12: 1481). The design of cyclic peptides, the conformation of which is suitable to bind MHC-molecules and to interact with T-cell-receptors in order to induce a strong immune response, is complex and thus laborious. Thus, for many of the important uses of peptide-based vaccines, e.g. cancer immunotherapy, no cyclic peptides could be generated and tested, yet (Lazoura and Apostolopoulos, Curr Med Chem, 2005, 12: 629).

Retro-inverso peptides were developed in order to mimic the structure and function of native peptides in the form of a molecular mimicry. Retro-inverso analogs of native peptides were produced using standard methods of peptide synthesis, whereby the direction of the synthesis of the peptide bonds is inverted, and D-amino acids are used instead of L-amino acids. Nevertheless, in addition the C-terminus of the retro-inverso peptide must be amidated, and the N-terminus must be acetylated, since otherwise the immunogenicity of the peptides is lost (Nair et al., J Immunol, 2003, 170: 1362). An advantage of retro-inverso peptides is that they are not degraded by the naturally occurring peptidases. Thus, retro-inverso peptides mainly exhibit a markedly higher stability in the body. When using retro-inverso peptides, the successes with respect to the antigenic and immunogenic properties were very different (Lazoura and Apostolopoulos, Curr Med Chem, 2005, 12: 629). Whilst, for example, an immune response could be achieved with a retro-inverso peptide from polio virus (PV1₁₀₃₋₁₁₅) which was comparable to the native peptide, this was not possible with retro-inverso analogs of other peptides, such as, for example, the toxin α₂₄₋₃₆, the HEL₁₀₃₋₁₂₁, the rep₁₂₋₂₆, and the OVA₃₂₃₋₃₃₉ (Herve et al., Mol Immunol, 1997, 34: 157, Meziere et al., J Immunol, 1997, 159: 3230).

In the development of peptide-based vaccines, the incorporation of non-naturally occurring amino acids in peptides is, for example, used, in order to increase the metabolic stability, but also for improving the lipophilic properties of the peptides, and thus to influence the binding properties of the peptides. Thus, for example, the stability of an EBV-epitope could be markedly increased through amidation of the C-terminus, together with a simultaneous efficient stimulation of cytotoxic T-lymphocytes. The exchange of the Met-Val bond by a CH₂—NH-linkage, or the attachment of a D-glucopyranosyl-unit to the threonine of the same EBV-epitope had a similar effect (Marastoni et al., J Med Chem, 2001, 44: 2370). The incorporation of β-amino acids instead of α-amino acids can also increase the stability of peptides against proteolytic degradation. Nevertheless, studies regarding effects of β-amino acids on the immunological properties of an epitope of the gp-120 protein from HIV showed that β-amino acids can negatively influence the T-cell-response (Poenaru et al., J Med Chem, 1999, 42: 2318).

The specific properties of peptides require, that, for the application of peptide-based vaccines, normally the use of adjuvants and/or carriers is required. In principle, the same adjuvants and carriers can be used for the application of peptide-based vaccines as can be used for classical vaccines. As examples, reference shall be made to complete and incomplete Freund's adjuvant, aluminum-based adjuvants, virosomes, liposomes, and proteasomes (Lazoura and Apostolopoulos, Curr Med Chem, 2005, 12: 1481).

In addition to the classical adjuvants and carriers, particular chemical methods were developed in order to produce peptide-based vaccines, and to be able to apply these. Here, a particular focus lies on techniques that allow to use peptides in vaccines as oligo- or polymers, in order to increase the immunogenicity.

In the MAP-system (Multiple Antigenic Peptide), peptides are chemically coupled to a core structure of lysines, in order to generate a multivalent vaccine (Tam, Proc Natl Acad Sci USA, 1988, 85: 5409). Comparatively high antibody-titers could be generated with MAP-constructs, which nevertheless as a prerequisite require the addition of adjuvant. Furthermore, the difficulty to generate MAP-products of high purity proved to be problematic (Olive et al., Mini Rev Med Chem, 2001, 1: 429). In addition, the number of different epitopes that can be integrated into the MAP-construct is very limited.

In order to increase the homogeneity of MAP-constructs, methods were developed by which the core matrix and the synthetic peptides can be separately synthesized and purified, and are ligated together in a final step. Using this, cyclic, linear and branched core structures can be generated that were used for the production of synthetic peptide-based immunogens (Mutter et al, J Am Chem Soc, 1992, 114: 1463, Nardin et al., Vaccine, 1998, 16: 590). This technology increased the flexibility during assembly of the synthetic epitopes, and thus allowed for a control of the orientation of the epitopes as well as of the overall structure of the molecule that can be decisive for the kind of the immune response by the immunogen. Despite the progresses in the production of highly purified epitope-based immunogens that could be obtained using this method, the relatively low number of epitopes that can be integrated into a molecule remains as a limitation (Olive et al., Mini Rev Med Chem, 2001, 1: 429).

An improvement of the MAP-system is the LCP-system (Lipid Polylysin Core Peptide). In this system, the immunogenicity of the synthetic peptides shall be increased by chemically coupling a lipid anchor to the polylysin core (Toth et al, Tetrahedron Lett, 1993, 34: 3925). The lipid anchor is coupled as lipid amino acid-part to the C-terminus of a polylysin-peptide-system. It could be shown with an LCP construct containing the synthetic peptides from the variable domain of the outer membrane proteins of Chlamydia trachomatis, that the immunogenicity of the synthetic peptides was markedly increased, compared to the immunogenicity of the same peptides when these were administered as monomers with adjuvant (Zhong et al., J Immunol, 1993, 151: 3728).

Based on the polymerization of synthetic peptides as induced by free radicals, a method was developed by Jackson et al. in order to produce an artificial, polyvalent protein containing a large number of identical or different epitopes (Jackson et al., Vaccine, 1997, 15: 1697). Here, synthetic peptides that were acylated with an acryloyl-group are polymerized, in order to produce an artificial molecule, wherein the synthetic peptides are attached as side chains on an inert alkane strand. Hereby, large synthetic molecules can be assembled, without the risk of synthesis errors that typically occur during the synthesis of long sequential molecules. Also using this approach, it could be shown that the immunogenicity of epitope-polymers is better than the one of epitope-monomers (Jackson et al., Vaccine, 1997, 15: 1697). Nevertheless, in practice it was found that, due to steric obstruction, the chemical polymerization is influenced by the size of the epitopes, and thus the introduction of a molecular spacer was required. Problems particularly also occurred during the production of heteropolymers, since the incorporation of the individual synthetic peptides into the alkane strand is random. Thereby, variations of the heteropolymers in between the individual syntheses are generated that affect the immunogenicity of the individual epitopes (Olive et al., Mini Rev Med Chem, 2001, 1: 429).

In summary, it remains to be noted that none of the currently available methods can fulfill all requirements that are essential in order to reproducibly and economically produce peptide-based vaccines, to simply and safely apply them to a human or animal, and that additionally can be safely and efficiently used in the clinic (Sette and Fikes, Curr Opin Immunol, 2003, 15: 461).

Thus, the invention is based on the problem that vaccines, which are based on short, unambiguously defined peptide antigens (≈antigenic determinants or epitopes), often exhibit only a very low immunogenicity when they are applied without adjuvants and/or carriers. In addition, most synthetic epitopes, when they are used for the immunization as monomers, have been found to be markedly less immunogenic, compared to epitope-polymers that function as antigens.

With the invention, a system for the production of vaccines is provided, wherein the vaccine forms a single unit of multimeric epitopes, adjuvant, and bacterial carrier. The multimeric epitopes that are targeted to be displayed on the surface of the bacteria in the following are designated as epitomers, in order to delimit them from conventional epitope-polymers. If targeted to and expressed on the surface of the host bacteria, and anchored on said surface, the epitomers prove to be effective antigens that induce an outstanding immune response. Following the immunization, a specific reaction of the immune system against the epitomer occurs. The invention thus allows for the production of peptide-based vaccines, wherein the orientation, number and combination of different epitopes in the epitomers can be freely chosen, and thus is clearly defined. In order to solve the object thereof, the invention therefore represents a new technology for increasing the low immunogenicity of peptide-based vaccines, and for producing a targeted epitope-based vaccine without the need to invest a lot of time and effort.

### Basic Concept of the Invention

The invention employs microorganisms, in particular bacteria, as production facilities for the antigens, as carriers of the antigens, and as adjuvants for the stimulation of the immune systems. By introducing nucleotide sequences into the genome of the bacteria, or into the cytosol in form of extrachromosomal DNA, the bacterium gains the following basic capacities: The bacterium expresses an antigenic determinant as oligomeric or multimeric epitope (the epitomer), and anchors said antigenic structure on the outer face of the bacterial cell wall. In addition, the carrier bacterium can be further genetically engineered or chemically modified regarding an improvement of the immune response.

One aspect of the invention therefore are bacteria that carry antigenic determinants in form of epitomers (oligomeric or multimeric epitopes) on the outer face of the bacterial cell wall. A further aspect of the invention is the use of these bacteria as a basis for a vaccine, or for any other kind of immunization, e.g. for the generation of monoclonal antibodies or for the production of polyclonal antisera.

In the following, the individual components are described that are required for the production of the epitomer-vaccination-bacteria.
(i) A nucleotide sequence encoding for at least one peptide sequence of an epitope of interest. An epitope in the sense of the invention generally consists of 5 to 40 amino acids. Nevertheless, also longer peptide sections can be used as epitopes. An epitomer consists of at least two units of an epitope that are genetically fused (e.g. epitope A in the constellation: A-A-A-A-A). The upper number of the epitopes is not limited (thus, only because of practical considerations). Nevertheless, the epitomer in the sense of the invention can also represent a series of two or more genetically fused epitopes (e.g. epitopes A, epitope B and epitope C: A-B-C-A-B-C-A-B-C) that are fused one to another in at least two consecutive units. The individual epitopes can either be fused directly, or can be linked one to another through a spacer sequence. The spacer sequence consists of at least one amino acid.
(ii) A nucleotide sequence encoding for a signal peptide which allows for the secretion of the at least one peptide sequence of the epitope, preferably the epitomer, through the cell wall of said carrier bacterium.
(iii) A nucleotide sequence encoding for an anchoring structure that attaches the at least one peptide sequence of the epitope, preferably the epitomer, on the outer surface of the carrier bacterium.
(iv) Optionally, a nucleotide sequence encoding for a spacer that is localized between the anchoring structure and the peptide sequence of the epitope, preferably the epitomer, and spans the cell wall. The spacer ensures that the epitomer is freely accessible on the outer surface of the carrier bacterium, and is not masked by the components of the cell wall.
(v) Nucleotide sequences allowing for the expression of one or any combination of the components (i) to (iv).

While retaining their function(s), the components (i) to (v) can be present in the bacteria according to the invention for one or more times in any combination.

Typical embodiments of the individual components as required for the production of epitomer-vaccination-bacteria according to the invention are described in more detail in the following.

### Component (i)

Component (i) is a nucleotide sequence encoding for an epitomer of interest, against which a specific immune response in the vaccinated individual shall be induced. Typical examples for epitopes that can be used in the form of epitomers on vaccination-bacteria are:
1. Epitopes of tumor-associated antigens: vaccination-bacteria with epitomers of tumor-associated antigens can be used for the prophylactic or therapeutic treatment of cancerous diseases. A multitude of tumor-associated antigens and specific epitopes that are recognized by T-cells have already been characterized, and are commonly known from publications and databases (e.g. www.cancerimmunity.org/statics/databases/htm).
2. Epitopes of antigens or pathogenicity factors of infectious pathogens: vaccination-bacteria with epitomers of antigens from infectious pathogens can be employed for the preventive or therapeutic immunization. The epitopes can, for example, be derived from antigens of bacteria, viruses, fungi or parasites, or can also be derived from mixtures of one or all of the given kinds of pathogens. Correspondingly, a single- but also a multiple-vaccination is achieved, when using epitomer-vaccination-bacteria. Exemplary epitopes are, for example, described in Goncharova et al., Int J Med. Microbiol., 2006; 296 Suppl 1: 195-201; Maillard and Pillot, Res Virol., 1998, 149: 153-61; Hervas-Stubbs et al., Infect Immun., 2006, 74: 3396-407; Cooreman et al., Hepatology, 1999, 30: 1287-92; Heijtink et al., Vaccine, 2001, 19: 3671-80; Ernst et al, Vaccine. 2006, 24: 5158-68; Koide et al, J Mol. Biol., 2005, 350: 290-9; Sehgal et al., Parasite Immunol., 2004, 26: 219-27; Lotter et al., J Exp Med., 1997, 185: 1793-801; Novotny et al., J. Immunol., 2003, 171: 1978-83; Srivastava et al., Hybridoma, 2000, 19: 23-31 and Woo et al., J. Virol., 2006, 80: 3975-84. These include epitopes from Hepatitis B, Streptococcus pneumoniae, Haemophilus, Entamoeba, Malaria, Borrelia, Influenza, Mycobacterium, and Encephalitis virus. Malaria-epitopes are described in, for example, EP 0 429 816. Additional epitopes are known to the person of skill.
2.3. Synthetic epitopes: vaccination-bacteria with entirely synthetic epitomers can, for example, be used for the generation of monoclonal and polyclonal antibodies that recognize peptide structures that do not occur naturally. Correspondingly, for example, antibodies for novel immunological tags can be produced that are of high value for molecular biology and protein biochemistry. Using this method, in addition novel enzymes can be generated whose enzymatic effects are triggered through the variable region of the antibody as generated.

The individual epitopes of the epitomer can be fused directly one to another, or can be separated by one or more spacers. The spacer should have a length of at least one amino acid, but will generally consist of 5 to 20 amino acids (or more amino acids). Within an epitomer, either always the same spacer can be used, or different spacers can be used. Important positions within the spacer, e.g. the first amino acid of the spacers following an epitope, can be optimized in order to achieve an optimal processing and presentation of the individual epitopes (Sette et al., Tissue Antigens, 2002, 59: 443). Respective methods are known to the person of skill.

### Component (ii)

Component (ii) is a nucleotide sequence encoding for a signal peptide that allows for the secretion of the epitomer from the carrier bacterium. The signal peptide is localized at the N-terminus of the epitomer-construct, and, in general, is between 15 to 35 amino acids in length (but can be longer in some cases). As a signal peptide, a naturally occurring, a genetically engineered and optimized, or an entirely synthetic idealized signal peptide can be used. As an example for naturally occurring signal peptides, the signal peptides of the InlA, InlB, and Lmo2714 from Listeria monocytogenes shall be mentioned (Cabanes et al., Trends Microbiol, 2002, 10: 238). The person of skill is aware of additional analogous and effective signal peptides.

Component (iii)

Component (iii) is a nucleotide sequence encoding for an anchoring structure that attaches the epitomer covalently or non-covalently on the outer surface of the carrier bacterium. The anchoring structure can be localized in the N-terminal region or at the C-terminus of the epitomer-construct. Naturally occurring, genetically engineered optimized or entirely synthetic anchoring structures can be used as anchoring structures. Depending from the carrier bacterium, the anchoring mechanisms of Gram-negative or Gram-positive proteins are used. Examples that can be used for Gram-negative carrier bacteria are the anchoring structures of (1) outer membrane proteins (e.g. OmpA, OmpC or PhoE), (2) lipoproteins (e.g. TraT, PAL or Inp), (3) autotransporters (e.g. VirG or AIDA-I), and (4) S-layer proteins, such as, for example, RsaA. Examples that can be used for Gram-positive carrier bacteria are the anchoring structures of (1) LPXTG-proteins (e.g. InlA, InlE, and Lmo2714), (2) proteins with GW-modules (e.g. InlB, Ami, and Lmo1076), (3) proteins with hydrophobic tail (e.g. ActA, SvpA, and Lmo2061), and (4) lipoproteins (e.g. GbuC, TcsA, and OpuCC).

### Component (iv)

Component (iv) is a nucleotide sequence encoding for a spacer that is localized between the anchor and the epitomer. The spacer shall ensure that the epitomer will not be positioned inside the cell wall of the carrier bacterium, or will be masked by the components of the cell wall, but is freely accessible on the outer surface of the carrier bacterium. As spacers, all peptide fragments can be used that can be expressed and secreted in the carrier bacterium. Naturally occurring sequences that are optimized through genetic engineering, or entirely synthetic amino acid sequences can be used as spacers. Examples for naturally occurring spacer sequences are the peptide fragments localized N-terminally from the LPXTG-anchoring structure of InlA or Lmo2714 from Listeria monocytogenes. Additional spacer sequences can be derived from the sequences of other bacterial surface proteins, and are known to the person of skill.

### Component (v)

Component (v) comprises one or more nucleotide sequences that allow for the expression of one or any combination of the components (i) to (iv). The nucleotide sequences in general comprise at least one prokaryotic promoter, and a suitable ribosomal binding site for each gene to be expressed. The promoter can be constitutively active or can be inducible. Optionally, one or more of the nucleotide sequences can contain operator structures that allow for a regulation of the expression. In addition, optionally one or more of the nucleotide sequences can encode for a regulator that regulates the expression of one or any combination of the components (i) to (iv). The nucleotide sequences of the component (v) can be naturally occurring, can be a sequence that is optimized by genetic engineering, or can be an entirely synthetic sequences. An example for such a nucleotide sequence is the promoter of the gene for Listeriolysin, including the prfA-box as the operator in combination with the 5′-untranslated region, and the ribosomal binding site of the gene for listeriolysin, in connection with a nucleotide sequence encoding for the regulator prfA that is under the control of its natural promoter. All components as mentioned are derived from Listeria monocytogenes. Other nucleotide sequences that allow for an expression of an epitomer are known to the person of skill.

All bacterial species can serve as carriers for the components (i) to (v) which can be manipulated using methods of genetic engineering. Apathogenic, pathogenic or bacteria that are attenuated in their pathogenicity can be used as carrier bacteria. Furthermore, the carrier bacteria can be manipulated by genetic engineering or can be manipulated chemically, in order to improve the immune response. Thus, for example, immune stimulating proteins, such as, for example, interleukins, chemokines, cytokines or interferons can be produced by the carrier bacterium in addition to the components (i) to (v). These immune stimulating proteins can be present in the cytoplasm of the carrier bacterium, can be secreted into the surrounding, or can be anchored on the outer surface of the bacterium.

Thus, epitomer-vaccination-bacteria according to the invention are bacteria that are genetically engineered or chemically modified in view of an improvement of the immune response, and which, through the introduction of nucleotide sequences, have been provided with the ability to anchor epitomers on their outer surfaces. Furthermore, the use of these epitomer-vaccination-bacteria for any kind of immunization for a targeted induction of an immune response against the anchored epitomer is regarded as matter of the invention. The use of the epitomer-vaccination-bacteria can take place both in humans and in animals.

A further aspect of the invention are preparations that are used as medicaments, and, according to the invention, contain epitomer-vaccination-bacteria. Epitomer-vaccination-bacteria can be contained in these medicinal preparations that (i) carry a particular epitomer on carrier bacteria and belong to one single bacterial species, or that (ii) carry different epitomers on carrier bacteria and belong to one single bacterial species, or that (iii) carry a particular epitomer on carrier bacteria and belong to different bacterial species, or that (iv) carry different epitomers on carrier bacteria and belong to different bacterial species, or that (v) carry different epitomers on one carrier bacterium.

The epitomer-vaccination-bacteria can be used as living bacteria or as killed or inactivated bacteria. For a killing and/or inactivation of the vaccination-bacteria, any techniques for an inactivation of microorganisms that is known to the person of skill can be used.

The invention shall now be further explained in the following based on the examples with reference to the accompanying Figures, without being limited thereto. In the Figures:

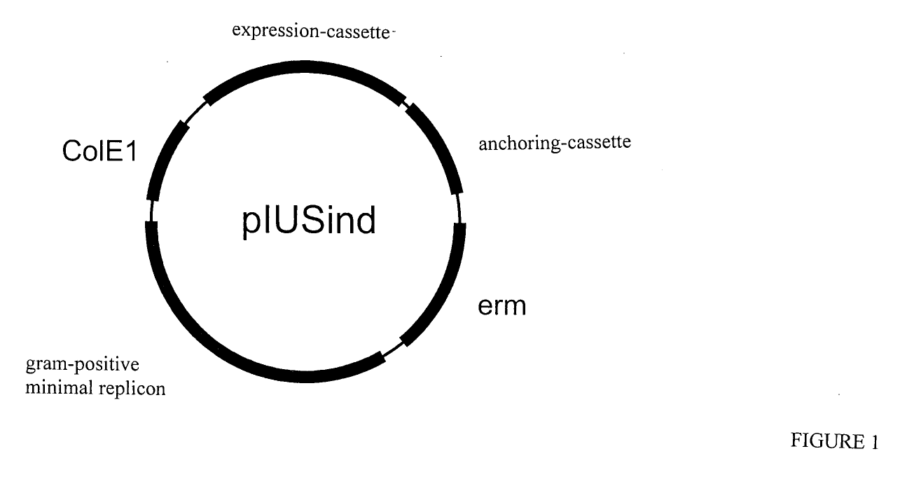

FIG. 1: shows a schematic outline of the vector pIUSind. ColE1: Origin of replication for E. coli; erm: Erythromycin resistance gene. Gram-positive minimal replicon: origin of replication for Listeria.

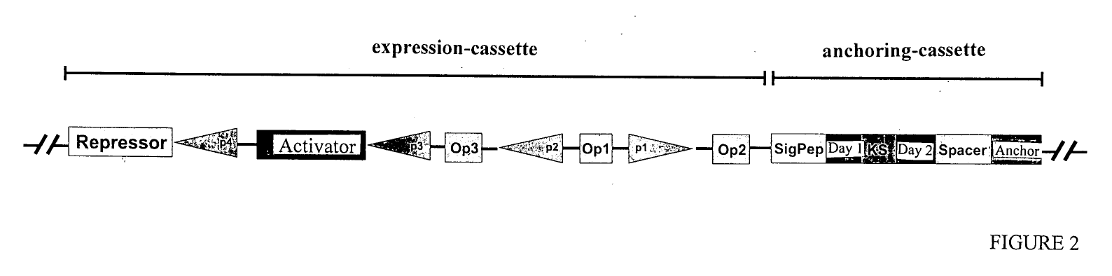

FIG. 2: shows a schematic outline of the expression- and anchoring-cassette of the vector pIUSind: Repressor: LacI-gene (E. coli); p4: constitutive promoter, native promoter of the p60-gene (L. monocytogenes); activator: prfA-gene (L. monocytogenes); p3: native promoter of the prfA-gene; Op1: prfA-box (L. monocytogenes); Op2 and Op3: LacO operator structures (E. coli); p2: promoter of the plcA-gene (L. monocytogenes); p1: promoter of the llo-gene (L. monocytogenes); SigPep: signal peptide of listeriolysin (L. monocytogenes). Tag1: S-tag; KS: cloning-restriction site; Tag2: myc-tag; Spacer: spacer for spanning the cell wall, partial fragment of InlA (positions 677-766); anchor: LPXTG-anchor from InlA (positions 767-800) (L. monocytogenes).

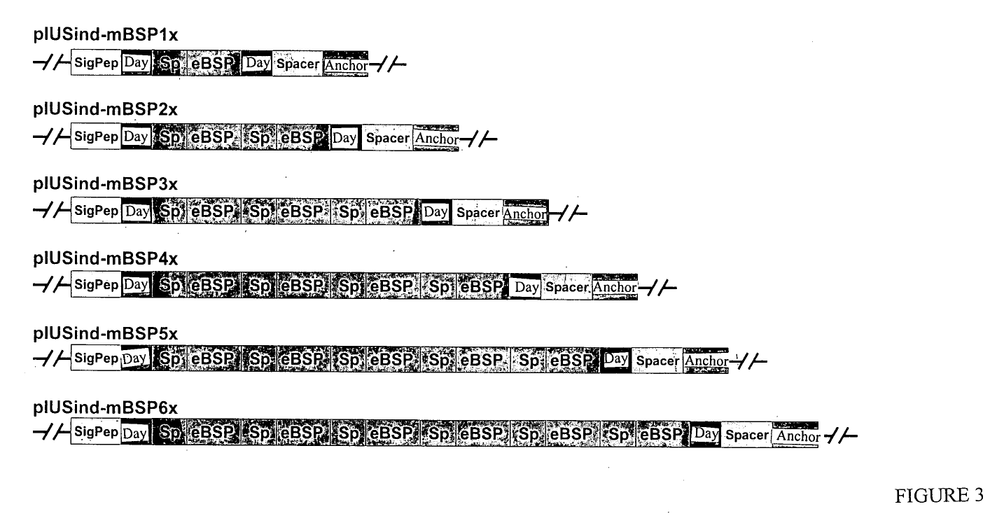

FIG. 3: shows a schematic outline of the anchoring cassette: SigPep: signal peptide of listeriolysin (L. monocytogenes); Tag: S-tag or myc-tag, respectively; Sp: spacer between the individual BSP-tumor epitopes (-IPSGGGGSA-); eBSP: BSP-tumor epitope (-EDATPGTGYTGLAAIQLPKKAG-); Spacer: spacer for spanning the cell wall, partial fragment of InlA (positions 677-766); anchor: LPXTG-anchor from InlA (positions 767-800) (L. monocytogenes).

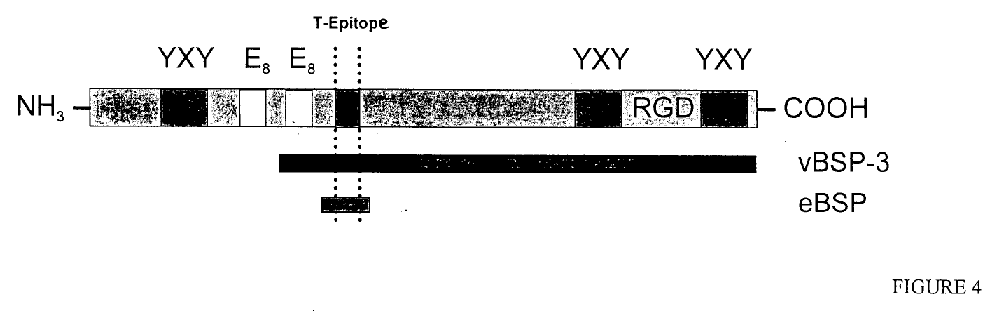

FIG. 4: shows a schematic outline of the BSP-molecule as well as the BSP-fragments vBSP3, and eBSP (the tumor epitopes), that were anchored on L. innocua. RGD: cell-binding motif of BSP; YXY: tyrosine-rich region; E8: glutamate-rich region; T-epitope: tumor-epitope.

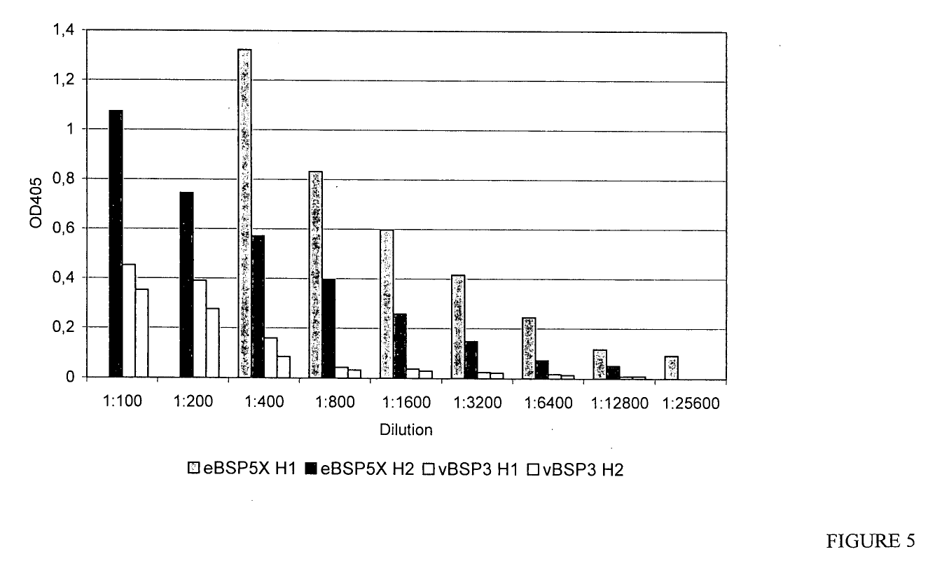

FIG. 5: ELISA for a determination of the antibody titer of rabbits that were immunized with pIUSind-eBSP5x or pIUSind-vBSP3 vaccination-bacteria. The titer of the serum was tested in a direct ELISA against a recombinant BSP-fragment as antigen.

### EXAMPLE 1

### Induction of an Immune Response Against a Tumor Specific Epitope of Bone Sialoprotein

The bacteria according to the invention are particularly suitable for the immunization against specific tumor epitopes that are known to the person of skill. As a preferred example, the method shall be described based on the tumor epitope of bone-sialoprotein (BSP). BSP is a 65 kDa highly glycosylated protein which is naturally found in bone, cartilage, and dentin. Compared to BSP from non-transformed cells, the tumor specific BSP exhibits a reduced glycosylation of a specific key position. This altered site of the tumor specific BSP in the following is referred to as BSP-tumor epitope. In the following the production of BSP-vaccination-bacteria and their use for the induction of BSP-specific antibodies is described.

### Generation of the Vector for the Anchoring

### Basic Design of the Vector

Listeria innocua was used as carrier bacterium for the BSP-epitomer. For anchoring of the BSP-epitomer, the plasmid pIUSind was generated (see FIG. 1). pIUSind is a shuttle plasmid containing origins of replication for E. coli and Listeria innocua. pIUSind contains an erythromycin resistance gene (erm) as a resistance marker.

### Expression Cassette

In order to achieve the expression of epitomers in L. innocua, an expression cassette was integrated into pIUSind. All components of the expression cassette were amplified with PCR from the organisms as indicated, and cloned using molecular biological techniques that are known to the person of skill. The arrangement of the individual components of the expression cassette on the plasmid can be taken from FIG. 2.

The promoter of the gene for listeriolysin from Listeria monocytogenes was selected (p1) for the expression of the epitomers. The promoter p1 is under the control of the operator structures Op1, Op2, and Op3. Op1 is a prfA-Box from L. monocytogenes, and is used for the positive regulation of the p1 promoter. Op2 and Op3 are LacO-operator structures from E. coli that allow for a repression of the promoter p1. Upstream of the operator Op3 the prfA-gene is integrated as an activator under the control of its native promoter from L. monocytogenes. PrfA functions via the operator Op1, and activates the promoter p1. The lacI gene from E. coli under the control of a promoter that is constitutively activated in L. innocua is integrated upstream of the prfA-gene as repressor. LacI functions via the operator structures Op2 and Op3, and represses the promoter p1. The repression of the p1 can be removed by LacI through the addition of IPTG. The combination of LacI, p1, Op2, and Op3 thus allows for an inducible expression of a gene in L. innocua.

### Anchoring Cassette

In order to achieve the anchoring of epitomers on L. innocua, the following components were introduced into the expression cassette. Downstream of the listeriolysin promoter, coding sequences for the signal peptide of listeriolysin (SigPep) and for two immunological tags (tag1 and tag2) were integrated. Between the immunological tags, a restriction site for cloning (KS) is found that allows for the integration of nucleotide sequences encoding for epitomers of different copy-number (here 1 to 6-fold). Structures from the InlA (Swiss-Prot: Q723K6) were used as anchor and spacer for the epitomer. Directly downstream of tag2, nucleotide sequences were integrated encoding for the peptide fragments of positions 677-766 (spacer) and 767-800 (anchor) of InlA, respectively.

### Generation of the Anchoring Plasmid for the BSP-Epitomer

For the anchoring of BSP-tumor-epitomers on Listeria, different anchoring plasmids were generated. In the restriction sites for cloning between both immunological tags, the anchoring plasmids contained nucleotide sections encoding for the BSP-tumor epitope as monomer or as an epitomer with 2 to 6 copies of the BSP-tumor epitope. The monomeric BSP-tumor epitope has the amino acid sequence -EDATPGTGYTGLAAIQLPKKAG- (eBSP). In the present example, the individual tumor epitopes are linked with another through a short spacer having the amino acid sequence -IPSGGGGSA- (Sp). Tumor epitope and spacer were amplified with PCR and were cloned into the anchoring plasmid pIUSind using standard techniques. A schematic representation of the anchoring cassettes of the anchoring plasmids as generated is shown in FIG. 3. The constructs for anchoring the epitomer pIUSind-eBSP1x-6x as generated allow for the covalent anchoring of the BSP-tumor epitope as monomer (pIUSind-eBSP1x) or epitomer in 2 to 6 copies (pIUSind-eBSP2x-6x) on Listeria spp, in particular L. monocytogenes and L. innocua.

Anchoring of Epitomers on L. innocua

The plasmids pIUSind-eBSP1x-6x were transformed into L. innocua cells by protoplast transformation. The success of the transformation was verified through preparation of the plasmid-DNA from the transformed L. innocua clones and restriction digestion of the prepared plasmid-DNA.

For anchoring of the BSP-tumor-epitomers, 5 ml brain heart infusion cultures containing 5 mg/L erythromycin (BHIₑᵣₘ-cultures) were each inoculated with an individual colony of Listeria clones pIUSind-eBSP1x-6x, and incubated over night at 37° C. with shaking. On the next morning, 5 ml BHIₑᵣₘ-cultures containing 1 mM IPTG were inoculated 1:20, and incubated for 4 h at 37° C. with shaking. During this period, the bacteria expressed the BSP-tumor-epitomers and anchored them on their cell walls. The detection of the successful expression of the BSP-tumor-epitomers on the surface of the bacteria was performed by flow cytometry using immune fluorescence-staining (Data not shown).

### Production of BSP-Vaccination-Bacteria

For the immunization of rabbits, BSP-vaccination-bacteria with the clone L. innocua pIUSind-eBSP5x were produced. For this, a 5 ml BHIₑᵣₘ-culture was inoculated, and incubated 3 h at 37° C. with shaking. The pre-culture was centrifuged, the bacterial pellet was resuspended in 10 ml of a synthetic minimal medium containing 5 mg/L erythromycin, and incubated over night at 30° C. with shaking. Subsequently, the BSP-vaccination-bacteria were inactivated by the addition of formaldehyde (1% final concentration, 24 h at room temperature). The quality of the BSP-vaccination-bacteria was ensured using a characterization of the inactivated vaccination-bacteria by flow cytometry. Measurements of the inactivated BSP-vaccination-bacteria showed that the inactivation of the vaccination-bacteria had no negative effects on the anchoring of the BSP-tumor epitope-polymer on the Listeria (Data not shown).

Immunization with BSP-Vaccination-Bacteria

Initially, rabbits were each immunized subcutaneously with a dosage of 109 inactivated pIUSind-eBSP5x bacteria without the addition of adjuvant, and were subsequently boosted in intervals of 4 weeks with the same dosage of vaccination-bacteria. As a control, rabbits were immunized with BSP-vaccination-bacteria carrying a larger partial fragment of the BSP (vBSP3) that was anchored on the surface. The vBSP3 fragment comprises the positions 84aa-318aa of the BSP-molecule (see FIG. 4).

Sera were obtained from the immunized rabbits by venous puncture, and the antibody titer against recombinant BSP was subsequently determined in an ELISA.

Surprisingly, the immunization with eBSP5x-vaccination-bacteria was found to be much more effective compared to the comparative immunization with vBSP3 (see FIG. 6). Whereas the animals that were immunized with eBSP5x-vaccination-bacteria had a titer of 1:1600 to 1:6400, the serum of the rabbits immunized with vBSP3 merely reached a titer of 1:200.

In an additional experimental setting, the immunogenic effect of the BSP-vaccination-bacteria was also tested in rats. Again, the animals were immunized either with eBSP5x- or with vBSP3-vaccination-bacteria. Also in this animal model, a comparable result could be found. Again, the animals reacted on the immunization with eBSP5x- vaccination-bacteria with a markedly stronger immune response than to the immunization with vBSP3 (Data not shown). The immunized rats subsequently served as a basis for the production of hybridoma cell lines producing BSP-specific antibodies. For the production of the hybridoma cells, the rats were killed, the spleens were prepared, and spleen cells were fused with the cells of a mouse-myeloma cell line using methods known to the person of skill. By screening of the cell supernatants, subsequently those hybridoma cells were isolated, that produced antibodies against BSP. It was realized that monoclonal antibodies against the BSP-tumor epitope could only be generated from the rats that were immunized with the eBSP5x-vaccination-bacteria.

The result of the immunization using eBSP5x-vaccination-bacteria is particularly surprising, since the BSP-tumor epitope is a very small protein fragment of only 22 amino acids, which was coupled on the bacterial surface as fivefold epitomer. In contrast, the vBSP3-fragment has a size of 234 amino acids. In contrast to the common opinion, thus an effective immune response was not induced against the large fragment, but against the epitomer. In addition, Listeria have a multitude of surface-proteins (133 in case of Listeria monocytogenes). It is therefore surprising that in particular a small, additionally added protein triggers a specific and efficient immune response.

The assumed reason for the high immunogenicity of the vaccination-bacteria according to the invention is the fact that the epitope is present on the surface of the vaccination-bacteria in multiple consecutive copies. The result shows that, using the vaccination-bacteria according to the invention, a specific, high and fast immune response can be induced.

An additional advantage of the bacteria according to the invention is that, through the targeted expression of a defined epitope, antibodies can be induced that are specific against this epitope. Whereas during the immunization with vBSP3-vaccination-bacteria antibodies are induced against the overall protein, the results of the inventors show that only with the aid of the vaccination-bacteria according to the invention, a targeted immune response against a specific epitope can be achieved.

In contrast to vBSP3, the tumor epitope effectively functions as an antigen, if secreted and bound on the surface in the form of epitomer-bacteria. Epitomer-bacteria thus exhibit an important advantage compared to other approaches of immunization with antigenic structures.

The experiments as performed show that bacteria, and specifically bacteria of the specie L. innocua, are suitable as carriers of epitomers and that an immune response can be induced with anchored epitomers. Furthermore, the experiments show that, when using epitomer-bacteria according to the present invention for the induction of an immune response, no further addition of adjuvant is required. In addition, it could also been shown that for the immunization vaccination-bacteria can be used that were killed using formaldehyde. Killed bacteria do not represent genetically modified organisms (GMO in the sense of the legal regulations), since they are no longer viable. This means that for the immunization according to the invention as described here, no GMO has to be used. The avoidance of the use of living GMOs in the vaccine is a decisive advantage in the registration of medicaments and vaccines.

1. A method for producing a carrier microorganism, the method comprising the steps of:
a) providing nucleic acid molecules, comprising
i) a nucleotide sequence encoding for at least one peptide sequence of an epitope of interest,
ii) a nucleotide sequence encoding for a signal peptide that allows for the secretion of the at least one peptide sequence of the epitope through the cell wall of said carrier microorganism,
iii a nucleotide sequence encoding for an anchoring structure that attaches the at least one peptide sequence of the epitope on the outer surface of said carrier microorganism,
iv) optionally, a nucleotide sequence encoding for a spacer being localized between said anchoring structure and the peptide sequence of the epitope, and spanning the cell wall, and
v) one or more nucleotide sequences that cause the expression of the nucleotide sequences i-iv in the carrier microorganism,
b) providing a carrier microorganism, and
c) introducing the nucleic acid molecules into the carrier microorganism, so that said microorganism suitably expresses the nucleotide sequences.

2. The method according to claim 1, wherein the carrier microorganism is selected from a bacterium, a fungus, in particular yeast, or a mammalian cell.

3. The method according to claim 1, wherein the nucleotide sequences (i)-(v) are present in the carrier microorganism one or more times in combination.

4. The method according to claim 1, wherein the nucleotide sequences (ii)-(v) are derived from the carrier microorganism or another organism.

5. The method according to claim 1 wherein the peptide sequence of an epitope of interest comprises about 5 to 40 amino acids.

6. The method according to claim 1, wherein the at least one peptide sequence of the epitope of interest comprises an epitomer.

7. The method according to claim 6, wherein the epitomer is present in the form of at least two epitopes of interest that are genetically linked one to another.

8. The method according to claim 6, wherein epitopes in said epitomer are present directly fused one to another, or are linked one to another by a spacer sequence.

9. The method according to claim 8, wherein the spacer sequence consists of at least one amino acid and/or chemically modified amino acid.

10. The method according to claim 6, wherein the epitomer comprises at least two identical units of an epitope that are linked one to another.

11. The method according to claim 6, wherein the epitomer exclusively comprises identical units of an epitope that are linked one to another.

12. The method according to claim 6, wherein the epitomer represents a sequence of different epitopes that are genetically linked one to another, which are present in at least two consecutive units.

13. The method according to claim 6, wherein the epitomer comprises about 1 to 10 epitopes.

14. The method according to claim 6, wherein the epitomer is present in about 2 to 10 copies.

15. The method according to claim 1, wherein the nucleotide sequences are introduced into the genome of the carrier microorganism, or introduced as extrachromosomal nucleic acid.

16. The method according to claim 1, wherein the nucleotide sequences are introduced by means of transformation, transfection and/or electroporation.

17. The method according to claim 1, wherein the nucleotide sequences are present on one or more genetic constructs.

18. The method according to claim 1, wherein the nucleotide sequences are present on one or more plasmids or one or more anchoring cassettes.

19. The method according to claim 1, wherein the nucleotide sequence (ii) is selected from the nucleotide sequence which essentially encodes for the signal peptide of listeriolysin.

20. The method according to claim 1, wherein the nucleotide sequence (iii) comprises the nucleotide sequence which essentially encodes for the LPXTG-anchor from the InlA having the positions 767-800 from L. monocytogenes.

21. The method according to claim 1, wherein the nucleotide sequence (iv) comprises the nucleotide sequence which essentially encodes for the partial fragment of the InlA having the positions 677-766.

22. The method according to claim 1, further comprising a further genetically engineered and/or chemical modification of the carrier microorganism for an improvement of the triggering of the immune response.

23. A method for producing a pharmaceutical composition, comprising the method according to claim 1, and formulating the carrier microorganism with a pharmaceutically acceptable carrier and/or excipient.

24. The method according to claim 23, wherein the pharmaceutical composition is a vaccine.

25. The method according to claim 24, wherein the vaccine further comprises a common adjuvant.

26. The method according to claim 23, wherein the carrier microorganism is killed.

27. Pharmaceutical composition, in particular vaccine, produced according to the method of claim 23.

28. A method for immunizing a mammal, comprising administering a pharmaceutical composition, in particular a vaccine, according to claim 27.

29. The method according to claim 28, wherein the immunization takes place in the context of the production of monoclonal antibodies or polyclonal antisera.

30. The method according to claim 28, wherein the immunization takes place in the context of the active immunization in cancer therapy.

31. A method for an improved treatment of cancerous diseases, comprising administering an effective amount a pharmaceutical composition, in particular a vaccine according to claim 27, to a mammal in need of this treatment.

32. The method according to claim 30, wherein said mammal is a human.

33. The method of claim 2, wherein said bacterium is a Listeria species.

34. The method of claim 33, wherein said Listeria species is L. innocua.

35. The method of claim 2, wherein said fungus is a yeast.

36. The method of claim 5, wherein said peptide sequence of an epitope of interest comprises about 5 to 30 amino acids.

37. The method of claim 5, wherein said peptide sequence of an epitope of interest comprises about 6 to 23 amino acids.

38. The method according to claim 14, wherein said spacer comprises the sequence IPSGGGGSA.

39. The method according to claim 13, wherein the epitomer comprises about 2 to 8 epitopes.

40. The method according to claim 13, wherein the epitomer comprises about 5 to 6 epitopes.

41. The method according to claim 14, wherein the epitomer is present in 6 copies.

42. The method according to claim 19, wherein the nucleotide sequence (ii) encodes the signal peptide of listeriolysin from L. monocytogenes.

43. The method according to claim 26, wherein the carrier microorganism is killed through chemical killing with formaldehyde or by radiation.

44. The method according to claim 30, wherein the cancer is a metastatic disease.

45. The method of claim 44, wherein the metastatic disease is bone cancer.

46. The method according to claim 31, wherein said cancerous disease is a metastatic cancerous disease.

47. The method according to claim 46, wherein the metastatic disease is bone cancer.


SEQUENCE LISTING (2 sequences)

SEQ ID NO: 1 - protein, 9 aa, Artificial Sequence
  Short Spacer (Sp)
IPSGGGGSA

SEQ ID NO: 2 - protein, 22 aa, H. Sapiens
EDATPGTGYTGLAAIQLPKKAG